(19) United States
(12) Patent Application Publication
(10) Pub. No.: US 2024/0350785 A1
(43) Pub. Date: Oct. 24, 2024

(54) APPARATUS AND METHOD FOR TREATING SUPERFICAL UP TO DEEP DERMAL HYPER PIGMENTATION MELASMA, ACNE AND ANTI-AGING FACIAL SKIN SEGMENTS

(71) Applicants: Chaim SHECHTER, Kfar Saba (IL); Or BISMUT, Jerusalem (IL); Arie WAINBERG, Kiryat Motzkin (IL); Ari Josef EICHENBAUM, Ein Ayala (IL)

(72) Inventors: Chaim SHECHTER, Kfar Saba (IL); Or BISMUT, Jerusalem (IL); Arie WAINBERG, Kiryat Motzkin (IL); Ari Josef EICHENBAUM, Ein Ayala (IL)

(21) Appl. No.: 18/685,568

(22) PCT Filed: Aug. 22, 2022
(86) PCT No.: PCT/IB2022/057853

§ 371 Date: Feb. 22, 2024

Related U.S. Application Data
(60) Provisional application No. 63/235,814, filed on Aug. 23, 2021.

(51) Int. Cl. A61M 37/00; A61M 5/42; A61N 5/06

(52) U.S. Cl. CPC A61M 37/0015; A61M 5/422; A61N 5/0616; A61M 2037/0023; A61M 2205/3303; A61M 2205/3313; A61M 2205/3375; A61M 2205/3606; A61M 2205/6054; A61N 2005/0661

(57) ABSTRACT

The subject matter discloses a detection unit (102) adapted for performing facial skin diagnose in a treated area and for providing a first measurement of skin layers thickness or for providing a second measurement of defected or infected skin tissue in a treated area; a microneedles array 1032 adapted for performing an injection process for treating said defected or infected skin tissue; in accordance with said first measurement or said second measurement; and a cooling system adapted for cooling down said treated area prior to said injection; said cooling down is for reducing pain.

### FIELD OF THE INVENTION

The present disclosure relates to medical devices in general, and to medical devices for treating from superficial to deep dermal hyper pigmentation (melasma), in particular and base on same treatment method to treat Acne & Anti-Aging (wrinkles & Skin Resurfacing) segments.

### BACKGROUND OF THE INVENTION

Melasma and/or Post Inflammatory Hyperpigmentation (PIH) treatment depends on the cause of the pigmentation. Therefore, for example in the hyper-pigmentation type Melasma which is expressed in brown pigmentation on the face, the treatment includes three main components:
Avoiding sun exposure.
Using sunscreens.
Using/treat with brightening substance such as hydroquinone, retinoic acid, kozic acid and/or vitamin C.
   In order to increase treatment efficiency, methods known in the art typically prepare a cosmeceuticals or drug that combines several substances in different concentrations. In stubborn cases it is necessary to increase the concentration of the active ingredients and even consider a series of chemical peels to remove the top layer of skin and improve the permeability of prepared active substance.
   Pigmentation Cause from Following Causes:
Women using birth control pills.
Pregnancy, and/or hormone therapy
Thyroid disease
Sun and exposure to UV light
Aging

### SUMMARY OF THE INVENTION

The term treated area refers herein to an area in the facial skin that has to be treated for superficial up to deep dermal hyper pigmentation melasma, acne and anti-aging facial skin segments.

One problem disclosed by the present disclosure is how to provide an effective and less painful treatment for Melasma or Post Inflammatory Hyper pigmentation (PIH), Acne & Anti-Aging (Wrinkles & Skin Resurfacing) facial skin segments. Since Melasma/Post Inflammatory Hyper pigmentation is not just a pigment buildup but a proliferation of melanocytes (cells that produce the pigment in the skin), the effectiveness of known in the art topical lightening preparations is very limited. There for methods known in the art treatment is by laser and are being escorted with pain and long healing time (“Down-Time”). On the other hand, the usage of chemical peeling or, or in some cases, the usage of liquid nitrogen is painful and also dangerous.

One solution is a method and device that cools down the treat area and that utilizes ultra-thin needles.

According to some embodiments the device includes 10 Mhz to 23 Mhz ultrasound frequency portable or non-portable dermal ultrasound unit. The ultrasound unit performs facial skin diagnose and provides a full facial skin analysis of skin and skin layers thickness. The ultrasound unit detects the damaged skin tissue and/or extra or less melanin fibers or any other skin phenomena detectable by Ultrasound system.

According to some embodiments the measurement values of the ultrasonic unit are used to accurate the cosmeceutical injection process into patient facial skin. According to some embodiment the injection process is performed by a disposable RFID protected cosmeceutical cartridge or syringe integrated with a disposable RFID protected injectable microneedles array with suitable shape for treated facial area.

According to some embodiments the device includes a cooling unit that reduce the treated area skin temperature to a temperature between 1.5° to 7° Celsius that reduces or eliminates the pain caused by the injection process.

According to some embodiment the system includes a disposable Microneedles cartridge and a disposable cosmeceutical cartridge both protected RFID to avoid the reusage of microneedles cartridge and avoid cosmeceutical cartridge refill or fakes.

One embodiment discloses by the present disclosure is an apparatus, the apparatus comprising: A detection unit (102) adapted for performing facial skin diagnose in a treated area and for providing a first measurement of facial skin layer thickness or for providing a second measurement of location or depth of defected or infected skin tissue in a treated area; a capsule (1035) comprising a cosmeceutical material; a microneedles array (1032) adapted for performing an injection process for injecting the cosmeceutical material for treating the defected or infected skin tissue in accordance with the first measurement or the second measurement; and a cooling system adapted for cooling down the treated area prior to the injection; the cooling down is for reducing pain. According to some embodiments the microneedles array 1032 comprises disposable needles. According to some embodiments an external diameter of the microneedle in the array being at least 0.2 millimeter. According to some embodiments the apparatus further comprising an RFID unit for protecting the microneedles array (1032). According to some embodiments the apparatus being for treating Hyperpigmentation (PIH). According to some embodiments the apparatus further comprising a CCD camera for capturing images of the treated area. According to some embodiments the apparatus is further adapted for treating a plurality of skin issues wherein the plurality of skin issues being one member selected from a group consisting of Acne Vulgaris, Acne scar, Wrinkles, Skin Resurfacing Superficial, dermal Hyperpigmentation and skin Filers wherein type of the microneedles array 1032 and type of the cosmeceutical material being in accordance with the skin issue. According to some embodiments the microneedle array comprises a plurality of microneedles. According to some embodiments the cooling system is adapted for cooling the skin area to 1.5 Celsius to 7 Celsius. According to some embodiments the capsule (1035) being disposable and RFID protected. According to some embodiments the apparatus further comprises UV356 nm UV Light (1022) is configured to lightening a HIP or Melasma spot of the treated area, the lightening is for enabling visual detection an operator.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1 shows a block diagram of a device for treating superficial up to deep dermal hyper pigmentation melasma, acne and anti-aging facial skin segments, in accordance with some exemplary embodiments of the subject matter;

FIG. 2 shows a block diagram of the detection unit, in accordance with some exemplary embodiments of the subject matter;

FIG. 3 illustrates skin measurement sample result made by Ultrasound unit, in accordance with some embodiments of the disclosed subject matter;

FIG. 4 illustrates Melasma/PIH 365 nm ultraviolet lamp detection result, in accordance with some embodiments of the disclosed subject matter;

FIG. 5 illustrates a block diagram of the treatment unit, in accordance with some embodiments of the disclosed subject matter;

FIG. 6 illustrates a top view of the treatment unit, in accordance with some exemplary embodiments of the disclosed subject matter; and

FIG. 7 shows a flowchart diagram of a method for superficial up to deep dermal hyper pigmentation melasma, acne and anti-aging facial skin segments, in accordance with some exemplary embodiments of the disclosed subject matter.

### DETAILED DESCRIPTION

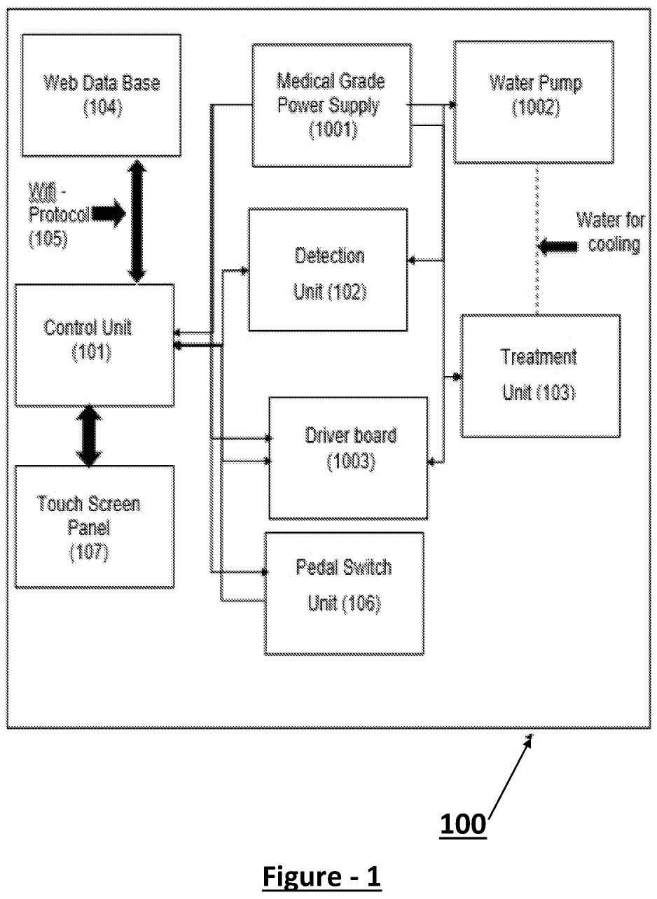

FIG. 1 shows a block diagram of a device for treating superficial up to deep dermal hyper pigmentation melasma, acne and anti-aging facial skin segments, in accordance with some exemplary embodiments

Device 100 is adapted for treating Hyperpigmentation from Superficial to deep dermal (Melasma) and/or PIH, Acne and Anti-aging (wrinkles & Skin resurfacing) skin segments and the like.

Device 100 comprises medical grade power supply (1001), Water Pump (1002), Custom Driver Board (1003), Control Unit (101), Detection Unit (102), Treatment Unit (103), WEB Data Base (104), Wi-Fi unit 105, Pedal Unit (106), and Touch Panel Screen (107)

The Medical grade power supply (1001) is adapted for supplying power. The Medical grade power supply (1001) receives entry electricity network 90-240 Vac 50/60 Hz and powers the following subsystem elements: Control unit (101), Detection system (102), Treatment Handpiece (103), Touch Screen (107), Water Pump (1002) and Driver board (1003).

The Medical grade Power supply 1001 can be, for example, CUS150Mby TDK-Lambda or any other similar power supply.

The Water Pump (1002) is adapted to supply water for cooling the cooling pads elements (not shown in the figure) that are located on Treatment unit (103). The pads elements cool down the patient treated area in order to reduce or to eliminate pain to the patient due to the injection process

The Water Pump (1002) is powered by the medical grade power supply (1001) and is controlled through the Driver board (1003) and control unit (101).

The water pump (1002) can be implemented by, for example, Winkeyes 400 Gph or Aquatec DDP5800 water pump or any other similar pump.

The Custom Driver Board (1003) is structured from several type of driver circuits on one board or several boards and is adapted to power and control the Main-system (100) the Water-Pump (1002), the Treatment-Unit (103), the Motor for capsule (1033) and the Motor for needles. (1034). The Custom Driver Board (1003) is further adapted to provide fractional RF (Radiofrequency) signals and Electro mesotherapy Rectangular Pulses for Treatment Unit (103) and the Disposable Needles array of the treatment unit.

The input side the Driver Board (1003) is connected to the Control-Unit (101) and to the Medical Grade Power Supply (1001). The output side of the Driver Board (1003) is connected to the device (100), to the Water Pump (1002), to the Treatment-Unit (103) to the Motor for capsule (1033), to the Motor for needles (1034) and to the Disposable micro Needles (1032) array (not shown in the figure)

The Control Unit (101) is configured for controlling and operating the entire device and sub system elements by Application software version

The Control Unit (101) is powered the Power-Supply (1001) and is connected to the Detection Unit (102), to the Treatment-Unit (103), to the Driver Board (1003), to the Touch Screen (107), to the Pedal-Unit (106) and to the Impedance-Sensor (1031) located on Treatment Unit (103)

The Control-Unit (101) can be implemented by utilizing, for example: a laptop, a Top-Desk PC or PC board. It can be Lenovo or similar vendor or PC board like Raspberry PI or similar.

The Detection Unit (102) is configured for analyzing patient skin condition using the Ultrasound (1021) system, UV365 UV light to analyze HIP cases and CCD Camera for collecting and documenting the information on WEB. The detection unit (102) is explained in greater details on FIG. 2,

The WEB Data Base (104) may be a Virtual data base that stores treated patient's information that is being collected by the device (100) during entire process stages (registration detection/analysis and treatment), based on patient username and password. The stored information on the WEB Data Base (104) can be pulled out upon operator request for analysis and other system application as needed. In some embodiments the process is complied with HIPPA (for USA market) & GDPR (for Europe market) privacy policy.

The WEB Data Base (104) receives and provides patient information to device (100) through internet protocol such as the Wi-Fi protocol (105).

The Pedal Unit (106) is a sub system that is being used as an operation electromechanical switch for the Treatment unit (103). The Pedal Unit (106) is connected to the Control Unit such that once the switch had been pressed, the signal is being transfer through the Control Unit (101) to the Treatment unit (103).

The Pedal Unit (106) can be, for example, TFS-201 Foot Switch Electric Momentary Pedal or any other.

It should be noted that the pedal unit (106) may be replaced by a manual push button which may be located on the treatment handpiece.

The Touch Panel Screen (107) is controlled and powered by Control Unit (101). The input of the Touch Panel Screen (107) receives power from the Control unit (101). The information that is presented on the Touch panel Screen (107) is received from the Control Unit (101).

The device (100) receives orders from the operator through the touch Panel Screen (107) and also present process indicators for instructing the user.

The Touch Panel Screen (107) can be, for example, Raspberry Pi 4B LCD capacitive 7-inch (or bigger) HDMI HD display USB drive or any other similar.

The treatment unit 103 is adapted for performing the injection process to Treat Melasma and/or HIP and or any other facial skin segment like Acne or Anti-Aging like wrinkles or skin resurfacing. The treatment unit 103 is explained in greater details in FIG. 5.

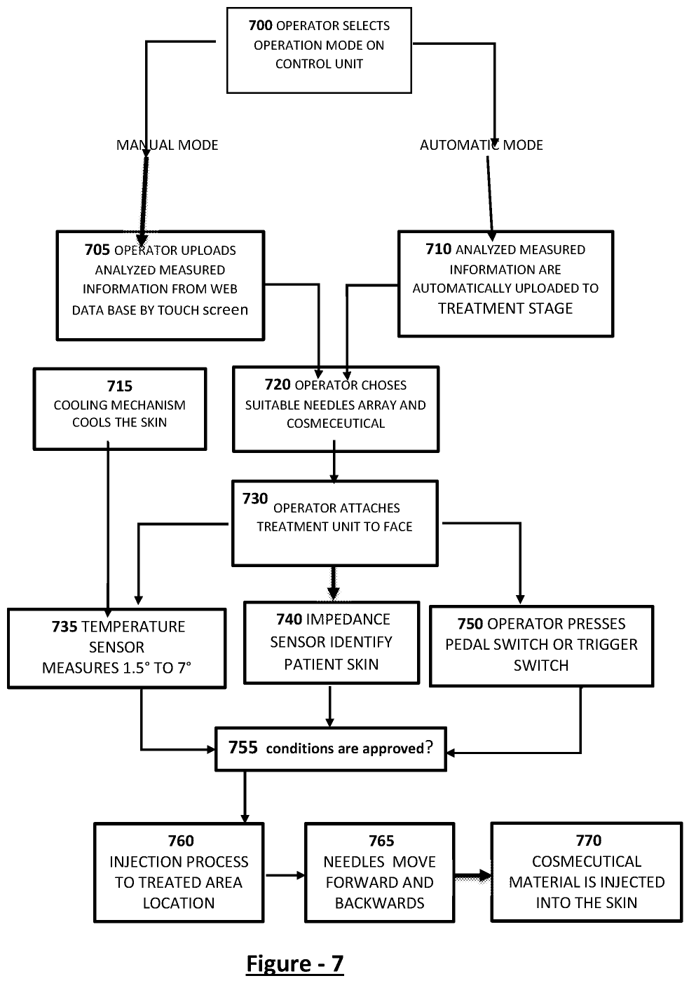

The device 100 can be operated in automatic mode or in manual mode. In manual mode all the operations are performed manually via the touch panel. The operation of the automatic mode is explained in FIG. 7.

FIG. 2 shows block diagram of the detection unit of the device for treating superficial up to deep dermal hyper pigmentation melasma, acne and anti-aging facial skin segments, in accordance with some exemplary embodiments of the subject matter.

The Detection unit 102 includes a CCD camera 1023, an ultrasound 1021 and a UV365 nm-390 UV light 1022.

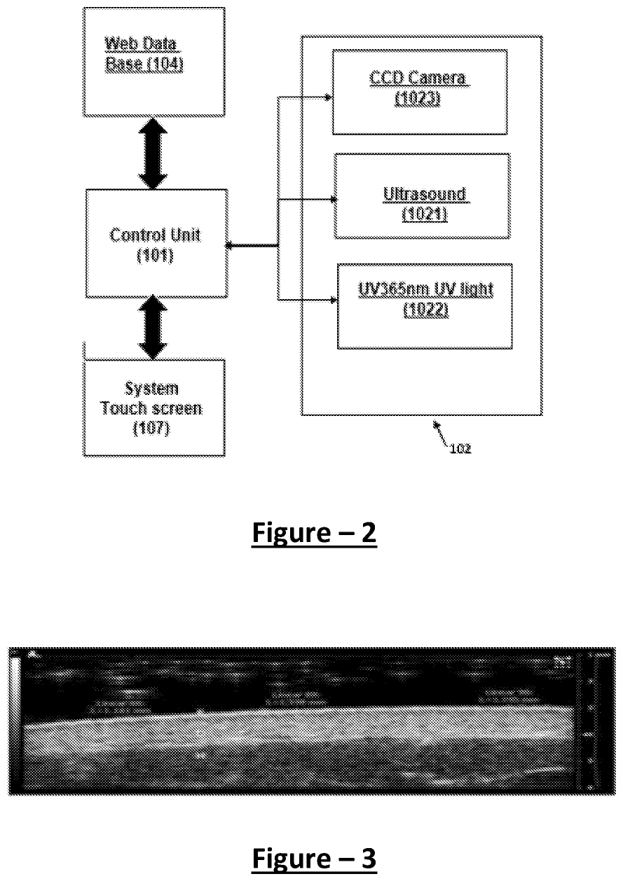

The Ultrasound (1021) is configured for scanning the designated treatment area, for measuring the skin and the skin layer and for detecting damaged skin tissues or extra melanin fiber location to define Superficial or dermal hyperpigmentation or any other facial skin phenomena like Acne or Anti-aging (Wrinkles & Skin structure). An example of extra melanin location is disclosed in FIG. 3. The detection process provides the reference values for the treatment process stage. The reference values include skin and skin layers thickness and information about damaged skin tissue like depth and size. The reference values are being stored on Web Data Base (104) under patient file defined by password and username and may be presented on the screen and are also transferred to the treatment unit for controlling the needles and cosmeceuticals operation.

The Ultrasound (1021) is connected to the control-unit (101) and is operated by an operation system. The Ultrasound (1021) is powered by the medical grade power supply in case of wired unit or by internal battery incase by wireless unit (not shown in this figure) and is controlled by the control unit (101).

The Ultrasound (1021) can be, for example a wireless Bluetooth connected 10 Mhz to 23 Mhz L20 HD Ultra-High Frequency Linear or similar wired or wireless models like the CLARIUS HD Portable Ultrasound Machine.

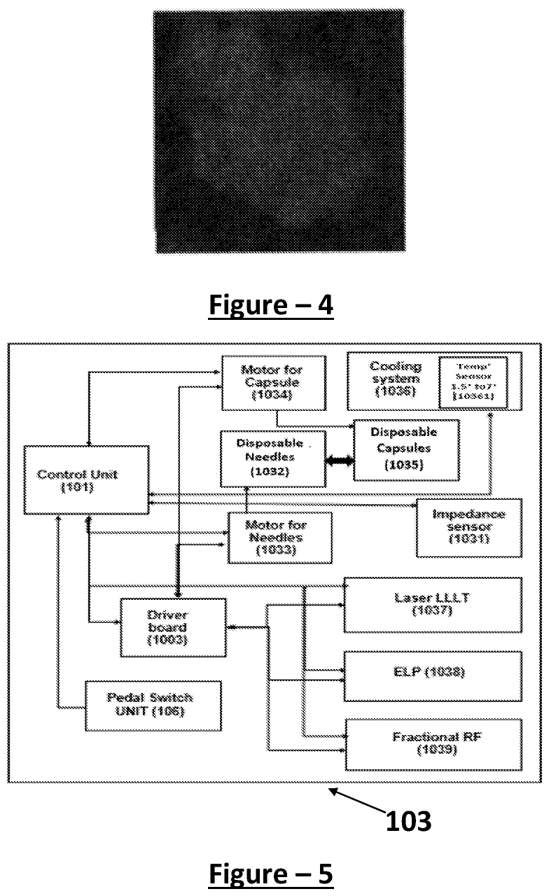

The UV356 nm UV Light (1022) is configured to lightening the HIP or Melasma spot as disclosed as an example in FIG. 4. The lightening is for enabling visual detection by the operator either in the superficial epidermis layer or in deep into Dermis layer or in between. The lightening is for enabling the capturing of the superficial epidermis layer or in deep into Dermis layer or in between. The UV365 nm to UV390 nm LEDs are powered and controlled from Control unit (101).

The UV365 nm UV light (1022) may be, for example, be eight units or more of LTPL-C034UVH365 or similar.

The CCD Camera (1023) is configured for collecting treated and measured area images and for storing the images under treated patient username & password for further analysis. The images may be transferred to the WEB Data Base (not shown in the figure) through the Wi-Fi protocol.

The images may also be presented to the operator. The CCD Camera (1023) is connected to the Control unit (101) and to the power supply for operation power. The CCD Camera (1023) is controlled by the control unit 101. The CCD Camera” (1023) can be for example Omnivision OVM9724 Color CMOS720 (1280×720) HD Camera CubeChip or similar.

The camera (1023) and the UV365 nm UV light (1022) are operated via the touch screen (107). The output of the camera (1023) and of the ultrasound (1021) is stored in the wen (104)

The camera (1023) and the UV365 nm UV light (1022) are placed on a treatment hand (not shown in the figure).

FIG. 3 illustrates the skin measurement sample result made by Ultrasound unit, in accordance with some embodiments of the disclosed subject matter;

FIG. 4 illustrates Melasma/PIH 365 nm ultraviolet lamp detection result, in accordance with some embodiments of the disclosed subject matter.

FIG. 5 illustrates a block diagram of the treatment unit in accordance with some embodiments of the disclosed subject matter; and

Treatment unit (103) includes Motor for capsule (1034), disposable cartridge 1035, motor for needles (1033), cooling system (1036), driver board 1003, Temperature Sensor (10361), disposable microneedles array (1032), impedance sensor (1031), laser LLLT (1037), ELP (1038), and fractional RF (1039).

The Impedance sensor (1031) is adapted for acting as one of the three safety interlock that enables the injection process of the Treatment unit (103) for allowing the treatment only when the treatment handpiece touches the skin, the Skin Temperature is between 1.5° to 7° Celsius as measured by Temperature sensor (10361) and the system identifies that the operator presses the pedal unit (106).

The impedance sensor (1031) measures the impedance of the facial skin for detecting that the treatment handpiece touches the patient facial skin.

The Temperature sensor (10361) verifies that the temperature of the skin is 1.5-7° Celsius. The identification is for preventing the start of the treatment when the temperature is above 1.5-7° Celsius.

The temperature sensor (10361) can be implemented by PT100 series sensors or similar.

The impedance sensor (1031) is connected to the control unit (101) and is adapted to touch the patient facial skin.

The Impedance sensor (1031) may be a mechanical custom-made part or any other type of part.

The Disposable microneedles Needles array (1032) cartridge is adapted to perform the injection process with needles thickness of 34 G (0.18 mm′) up to 22 G (0.4 mm′) or any needle that transfer the relevant Cosmeceutical Material that treats melasma or HIP Acne Wrinkles or skin resurfacing or any other anti-aging phenomena, from the Disposable capsule (1035) into patient facial skin or any other location decided the operation provider as long it does not contravene regulatory guidelines or warnings handling relevant product.

The Disposable microneedles array (1032) are assembled into the treatment unit (103). The Disposable microneedles array (1032) are being operated and connected to Motor for needles (1033). The Disposable micro needles array (1032) are custom made. The rage of used needles is thinner needle diameter (G=gage) of 0.18 mm′ (34 G) 0.23 mm′ (32 G) up to 28 G or any other diameter of hollow and non-hollow/isolated and non-isolated” needle's custom made. The Disposable microneedles array (1032) is located on a treatment hand (not shown in the figure).

The Motor for Needles (1033) is configured to move the Disposable micro needles array (1032) forward and backwards.

The Motor for needles (1033) is connected from one side to the Driver Board (1003) and to the Control Unit (101) and from the other side to the Disposable needles (1032).

The Motor for needles (1033) may be implemented by utilizing, for example, 12 Volts Maxon motor type: XD-25 G370 or any similar.

The Motor for Capsule (1034) is adapted for main pushing out the Cosmeceutical Material from the Disposable Cartridge (1035) through the microneedles array (1032) into patient treated area. The Motor for Capsule (1034) is connected from one side to the Driver Board (1003) and to the Control Unit (101) and from the other side to the Disposable cartridge (1035).

The Motor for Capsule (1034) may be implemented by, for example, utilizing 12 Volts Maxon motor type: XD-25 G370 or any similar.

The Motors for Capsule (1034) and Motor for needles (1033) are the same type and can be: 12 Volts Maxon motor type: XD-25 G370 or similar

The Disposable capsule (1035) includes a custom-made disposable capsule that contains Cosmeceutical Material. The Disposable capsule (1035) is for one treatment only and is protected from fakes (fake capsules) by RFID or similar method.

The Cosmeceutical Material includes cosmetic substance. The substance includes active ingredients per treated phenomena, that is being filled into the Disposable capsule (1035) to treat the Melasma and/or PIH, Acne or any other Anti-Aging or cosmetic phenomena.

The Disposable capsule (1035) is mounted on a designated place on the Treatment unit (103) and from the other side the material is being pushed out of the capsule with Motor of capsule (1034) and is injected through the Disposable Micro Needles array (1032) to designated treatment area on patient.

The Disposable capsule (1035) should be made of biocompatible injected plastic material

The Cooling system (1036) is adapted to cool down the treated area to a temperature that reduces pain. In one embodiment the temperature is between 1.5° to 7° Celsius degree to reduce the injection pain being made by Disposable Micro needles array (1032).

The Cooling system (1036) is connected to Driver board (1003) and to the Control Unit (101) from one side. The Cooling system (1036) is assembled into the Treatment unit (103) from the other side, in a designated place to maximize the effect.

The Driver Board (1003) provides control signals to the Water pump (1002). The water pump (1002) circulates the Cooling Mechanism fluid and is connected to the Control Unit (101). The Control Unit (101) provides control signal to Cooling mechanism (1036).

The Laser LLLT (1037) includes 520 nm green laser (or any other) to treat melasma and/or PIH phenomena by lightning designated treated area with 520 nm lease beam or any other wavelength.

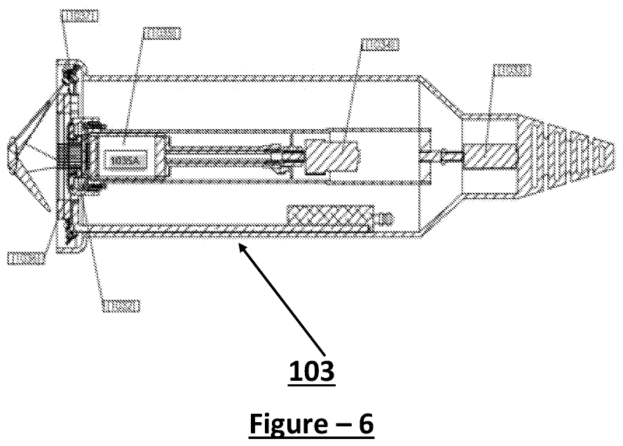

The Laser LLLT (1037) is connected from one side to Control Unit (101) for controlling signals. The Laser LLLT (1037) is connected to Drive Board (1003) for powering from the other side. The laser diodes are located on Treatment unit (103), on a certain place (see FIG. 6) to light most effectively the treatment area with the 520 nm or any other laser light.

The Laser LLLT (1037) may be, for example, Nichia 520 nm green diode or any other similar.

The ELP (1038) is adapted for providing rectangular electrical cyclic wave signals that treat Anti-Aging phenomena with the support of the Disposable micro-Needles array (1032) and relevant disposable capsule (1035) that can contain different active ingredients like Hyaluronic Acid or Vitamin A or any other approved cosmeceutical.

The ELP (1038) is controlled by the Control unit (101) and powered by Driver Board” (1003). The ELP (1038) output is connected to Disposable needles (1032) and disposable capsule (1032) sub system.

The Fractional RF (1039) is an RF (radio frequency) unit that provide RF signals on specific frequency and energy up to 62 mj/cm² or any other defined range that are begin transferred into patient facial skin through the Disposable Needles (1032) for cosmetic/anti-aging treatments.

The Fractional RF (1039) is connected from the input side to the Control-Unit (101) and to the Driver Board (1003). The Fractional RF (1039) is connected from the output side to the Disposable needles (1032) that transfer the RF signals into patient treated area.

FIG. 6 illustrates a top view of the treatment unit, in accordance with some exemplary embodiments of the disclosed subject matte

The treatment unit includes Disposable needles 1032, Motor for needles 1033, Motor for Capsule 1034, Disposable capsule 1035, Cooling system 1036, Laser LLLT 1037 and 1035a cosmeceutical material used for the treatment process. The cosmeceutical material is being filled into the cartridge (1035).

FIG. 7 shows a flowchart diagram of the method for treating Super Superficial to deep dermal Hyperpigmentation (melasma) Acne and Anti-Aging (wrinkles and skin resurfacing), in accordance with some exemplary embodiments of the disclosed subject matter.

At block 700 the operator starts the process by selecting the required operation mode on the Control unit (101).

Block 705 is performed if the operator selects the manual mode. At block 705 the operator uploads from the data base the data that has been collected by the detection process as well as other data that is associated with the treatment and with the patient.

Block 710 is performed if the operator selects the automatic mode. At block 710 the system uploads from the data base the data that has been collected by the detection process as well as other data that is associated with the treatment and with the patient.

At block 715 which is performed as a result of starting the process the cooling mechanism cools the skin of the treated area.

At block 720, the operator selects the needles and the cosmeceutical material that are relevant for the treatment.

At block 730, the operator attaches the Treatment Unit (103) to the facial skin of the patient.

At block 735 the temperature sensor senses the temperature of the treatment area.

At block 750 the operator presses system Pedal switch unit (106), or the trigger switch in order to start the operation.

At block 740 the impedance sensor (1031) located on the Treatment unit (103) check if the skin touches the treatment unit.

At block 755 the system check that all conditions for starting the treatment are met. The conditions are met if the temperature sensor senses that the temperature of the treatment area is between 1.5 Celsius to 7 Celsius degree and if the impedance sensor identifies that the treatment unit touches the treatment area and if the device identifies that the operator presses the pedal switch.

At block 760 the Disposable Needles (1032) array performs an injection operation by moving forward into the patient skin.

At block 765 the needles move forward with the support of Motor for needles (1033), that is connected to Driver board (1003), for operation signals and rated power. The motor pushes the disposables needles (1032) forward to penetrate the skin and pull backwards. According to some embodiments the needles (1032) movement speed is between 40 mm/sec up to 60 mm/sec though any other speed is applicable. While microneedles puling backwards the “Motor for capsule” (1034) relative to the type of the Motor for needles (1033) is connected to “Driver board” (1003) for control signals rated power.

At block 770 which is performed while the Disposable Needles (1032) are moving backwards. The Treatment Handpiece (103) pushes the cosmeceutical material through the Disposable needles (1032) into the skin layer.

1. An apparatus, the apparatus comprising:
A detection unit (102) adapted for performing facial skin diagnosis in a treated area and for providing a first measurement of facial skin layer thickness or for providing a second measurement of location or depth of defected or infected skin tissue in a treated area;
a capsule (1035) comprising a cosmeceutical material;
a microneedles array (1032) adapted for performing an injection process for injecting said cosmeceutical material for treating said defected or infected skin tissue in accordance with said first measurement or said second measurement;
and
a cooling system adapted for cooling down said treated area prior to said injection; said cooling down is for reducing pain.

2. The apparatus of claim 1, wherein said microneedles array 1032 comprises disposable needles.

3. The apparatus of claim 1, wherein an external diameter of said microneedle in said array being at least 0.2 millimeter.

4. The apparatus of claim 1 further comprising an RFID unit for protecting said microneedles array (1032).

5. The apparatus of claim 1 wherein said apparatus being for treating Hyperpigmentation (PIH).

6. The apparatus of claim 1 further comprising a CCD camera for capturing images of said treated area.

7. The apparatus of claim 1, wherein said apparatus is further adapted for treating a plurality of skin issues wherein said plurality of skin issues being one member selected from a group consisting of Acne Vulgaris, Acne scar, Wrinkles, Skin Resurfacing Superficial, dermal Hyperpigmentation and skin Filers wherein type of said microneedles array 1032 and type of said cosmeceutical material being in accordance with said skin issue.

8. The apparatus of claim 1 wherein said microneedle array comprises a single microneedle.

9. The apparatus of claim 1, wherein said microneedle array comprises a plurality of microneedles.

10. The apparatus of claim 1 wherein said cooling system is adapted for cooling said skin area to 1.5 Celsius to 7 Celsius.

11. The apparatus of claim 1 wherein said capsule (1035) being disposable and RFID protected.

12. The apparatus of claim 1 wherein said apparatus further comprises UV356 nm UV Light (1022) is configured to lightening a HIP or Melasma spot of said treated area, said lightening is for enabling visual detection an operator.
